(19) United States
(12) Patent Application Publication
(10) Pub. No.: US 2012/0045548 A1
(43) Pub. Date: Feb. 23, 2012

(54) METHOD FOR IMPROVING A BEAN-BASED PRODUCT

(75) Inventors: Leonardus Johannes Sofie Marie Mulleners, Rijen (NL); Pieter Cornelis Langeveld, Delft (NL); Wouter Willem Smits, Den Haag (NL)

(73) Assignee: DSM IP ASSETS B.V., Heerlen (NL)

(21) Appl. No.: 13/266,316

(22) PCT Filed: Apr. 21, 2010
(86) PCT No.: PCT/EP2010/055260

§ 371 Date: Oct. 26, 2011

(30) Foreign Application Priority Data
Apr. 28, 2009 (EP) 09158941.6

(51) Int. Cl. A23C 11/10; A23L 1/20; B65D 85/00; A23L 2/38; A23L 2/02; C12N 9/08; A23C 20/02

(52) U.S. Cl. 426/46; 435/192; 426/44; 426/629; 206/524.1

(57) ABSTRACT

The present invention relates to a method for improving at least one characteristic of a bean-based product by treating said bean-based product with a peroxidase, preferably a Marasmius scorodonius peroxidase or a functional equivalent thereof.

The present invention relates to a method for improving at least one characteristic of a bean-based product comprising contacting a bean-based product or an intermediate thereof with a peroxidase, preferably a Marasmius scorodonius peroxidase or a functional equivalent thereof, and optionally preparing from said intermediate a bean-based product.

### FIELD OF THE INVENTION

The present invention relates to a method for improving at least one characteristic of a bean-based product by treating said bean-based product or an intermediate thereof with a peroxidase, preferably a Marasmius scorodonius peroxidase or a functional equivalent thereof.

### BACKGROUND OF THE INVENTION

Beans in general and soybeans specifically are considered by many agencies, including the US Food and Drug Administration, to be a source of complete protein. A complete protein is one that contains significant amounts of all the essential amino acids that must be provided to the human body because of the body's inability to synthesize them. For this reason, soy is a good source of protein, amongst many others, for many vegetarians and vegans or for people who cannot afford meat.

According to the FDA, “Soy protein products can be good substitutes for animal products because, soy offers a “complete” protein profile. Soybeans contain all the amino acids essential to human nutrition, which must be supplied in the diet because they cannot be synthesized by the human body. Soy protein products can replace animal-based foods—which also have complete proteins but tend to contain more fat, especially saturated fat—without requiring major adjustments elsewhere in the diet.”

Unfortunately, food or beverages derived from or containing soy have some disadvantages, such as unpleasant taste, smell, color etc.

For example, soy milk is a beverage made from soybeans. A stable emulsion of oil, water and protein, which is produced by soaking dry soybeans, and grinding them with water. Soy milk contains about the same proportion of protein as cow's milk˜around 3.5%; also 2% fat, 2.9% carbohydrate and 0.5% ash. Tofu, soy milk “cheese”, is made by coagulating the protein from soy milk, just as cheese is made from, for example, cow milk. By appearance soy milk is different to cow milk and has pronounced yellow color as well as a characteristic soybean taste. In Asia, people are used to this taste, but in Europe and America, people do not like the soy bean taste and smell.

EP 0 328 322 A2 describes a composition for use in eliminating disagreeable odours from soya milk comprising glucose, glucose oxidase and, optionally, catalase. This publication further describes a process for the preparation of from soy milk free from disagreeable odors without detracting from gelation potential of the soy protein. The main effect described by this treatment is that dissolved oxygen in the water is almost completely eliminated. In the described method, catalase may be used for removing hydrogen peroxide produced by glucose oxidase.

U.S. Pat. No. 2,930,700 relates to improvements in the treatment of soy bean material, particular soy flour, for eliminating the undesirable taste or flavour which is characteristic of soy products. The flavour improving treatment involves subjecting the soy flour in aqueous medium to the action of yeast. Preferably, the yeast is utilized together with an added oxidizing agent or a selected alkaline neutralizing agent or both. It is speculated that the enzyme peroxidase in yeast is responsible in large measure for the elimination of the objectionable flavour of soy flour. The experimental data show that the treatment with yeast results in a significant increase in reducing sugar content thereby indicating that a substantial change occurs in the carbohydrate fraction of the soy flour. This increase in carbohydrate fraction results in flavour components which are strong enough to mask to a certain extent the objectionable flavors of soy flour. Catalase is used in U.S. Pat. No. 2,930,700, just as in EP 0 328 322, for removing excess of the oxidizing agent hydrogen peroxide.

### DESCRIPTION OF THE FIGURES

FIG. 1. Changes in b component of color during bleaching process, where control is not treated soy milk and samples: 1. only 1 mmol H₂O₂/I added, 2. 2.25 DBLU/ml+1 mmol H₂O₂/I, 3. 4.5 DBLU/ml+1 mmol H₂O₂/I and 4. 9 DBLU/ml+1 mmol H₂O₂/I.

FIG. 2. Changes in b component of color during bleaching process, where 1-12 samples are as described in example 2.

FIG. 3. Organoleptic results bleached soya milk, where 1-3 samples are as described in example 3.

FIG. 4. Changes in b component of color during bleaching process, where 1-6 samples are as described in example 5.

### SUMMARY OF THE INVENTION

As described above, bean-based products have disadvantages relating to their taste, flavour etc.

The inventors of the current invention have surprisingly found that the characteristics of bean-based products can be improved by incubating a bean-based product or an intermediate thereof with a Marasmius scorodonius peroxidase.

### DETAILED DESCRIPTION OF THE INVENTION

The present invention relates to a method for improving at least one characteristic of a bean-based product comprising contacting a bean-based product or an intermediate thereof with a peroxidase, preferably a Marasmius scorodonius peroxidase or a functional equivalent thereof, and optionally preparing from said intermediate a bean-based product.

The phrase “improving at least one characteristic” is used to refer to the situation that at least one characteristic such as taste or smell or colour (whiteness) (discussed in more detail later on) is improved compared to the situation in which said bean-based product or an intermediate thereof is not treated with a peroxidase, preferably a Marasmius scorodonius peroxidase or a functional equivalent thereof. The improvement can, for example, be measured based on results from a sensory panel and/or based on an analytical method.

The term “contacting” is used to refer to adding of a peroxidase, preferably a Marasmius scorodonius peroxidase or a functional equivalent thereof, to said bean-based product or an intermediate thereof. More preferably, this terms includes adding said peroxidase and allowing said peroxidase sufficient time to perform its activity.

When a method according to the invention is performed on a final bean-based product (for example a soy milk) the optional step of “preparing from said intermediate a bean-based product” can be omitted. In such a case the invention provides a method for improving at least one characteristic of a bean-based product comprising contacting a bean-based product with a peroxidase, preferably a Marasmius scorodonius peroxidase or a functional equivalent thereof.

However, when peroxidase, preferably a Marasmius scorodonius peroxidase or a functional equivalent thereof, is added to an intermediate of a bean-based product (for example to grinded beans), the invention preferably includes the step of “preparing from said intermediate a bean-based product”. In such a case, the invention provides a method for improving at least one characteristic of a bean-based product comprising contacting an intermediate of a bean-based product with a peroxidase, preferably a Marasmius scorodonius peroxidase or a functional equivalent thereof and preparing from said intermediate a bean-based product.

Reaction conditions, such as incubation temperature, incubation time, pH etc, can be easily determined by the skilled person without any undue experimentation. The preferred reaction conditions are based on the properties of the used peroxidase and preferably also on the features of the bean-based product or an intermediate thereof. For example, if the used peroxidase is inactivated at temperatures above 70 degrees Celsius, it is clear for the skilled person that the incubation temperature is preferably below 70 degrees Celsius. Preferably, the incubation conditions chosen are such that they are at or close to the optimas of the used peroxidase. It is clear that also the bean-based product or an intermediate thereof can have an impact on the treatment conditions. For example, if the used bean-based product or an intermediate thereof is unstable at low or high temperatures, such temperatures must be avoided. Preferably, treatment conditions are chosen as a compromise between the used peroxidase and the bean-based-product or an intermediate thereof.

As a guidance, the following ranges can be used.

In a preferred embodiment, the invention provides a method for improving at least one characteristic of a bean-based product comprising contacting a bean-based product or an intermediate thereof with a peroxidase, preferably a Marasmius scorodonius peroxidase or a functional equivalent thereof, and optionally preparing from said intermediate a bean-based product, wherein said bean-based product or an intermediate thereof is contacted with said peroxidase for a period of 1 minute to 5 days. Preferably, the incubation time is between 1 minute to 4 days or 1 minute to 3 days or 1 minute to 48 hours or 5 minutes to 48 hours or 10 minutes to 48 hours or 15 minutes to 48 hours or 20 minutes to 48 hours or 25 minutes to 48 hours or 30 minutes to 48 hours.

In yet another preferred embodiment, the invention provides a method for improving at least one characteristic of a bean-based product comprising contacting a bean-based product or an intermediate thereof with a peroxidase, preferably a Marasmius scorodonius peroxidase or a functional equivalent thereof, and optionally preparing from said intermediate a bean-based product, wherein said bean-based product or an intermediate thereof is contacted with said peroxidase at a temperature between 2 to 90 degrees Celsius. Preferably, the incubation temperature is between 2 to 85, 2 to 80, 2 to 75, 2 to 70, 2 to 65, 2 to 60, 2 to 55 or 2 to 50 degrees Celsius. More preferably, the incubation temperature is between 2 and 45 degrees Celsius. Even more preferred is a temperature range of between 15 to 40 or 25 to 40 degrees Celsius. Examples of suitable temperatures and incubation times are described within the experimental part.

The preferred combined incubation temperature and duration typically depend on each other. If for example, the optimum temperature of the used peroxidase is around 30 degrees Celsius and an incubation temperature of 8 degrees Celsius (assuming that the used particular peroxidase still shows some activity at this temperature) is chosen in view of the stability of the bean-based product or an intermediate thereof, it is clear that the incubation time should be increased compared to a situation in which the incubation temperature is 30 degrees Celsius. Thus, incubation temperatures deviating from the peroxidase optimum typically lead to an increased incubation time.

Examples of suitable incubation conditions are a temperature between 2 to 90 degrees Celsius and an incubation time of between 1 minute to 5 days.

It is furthermore possible to use multiple combined conditions in one and the same treatment. For example a first incubation for relative short period at higher temperatures, followed by an incubation for a relative longer period at lower temperature. For example, 2 to 3 hours at 15 to 40 degrees Celsius, followed by an incubation between 2 and 15 degrees Celsius for a period of 4 hours to 5 days.

The herein exemplified and preferred Marasmius scorodonius peroxidase has a temperature optimum of about 55 degrees Celsius (at least on beta-carotene) and its activity remains intact up to 60 degrees Celsius. At 70 degrees Celsius its activity is reduced to near zero. Hence, an incubation with the Marasmius scorodonius peroxidase is preferably performed at a temperature between 2 to 65, preferably between 2 to 60 degrees Celsius.

As will be shown within the experimental part, a peroxidase, preferably a Marasmius scorodonius peroxidase or a functional equivalent thereof, alone (when added in an effective amount and allowed to incubate under appropriate conditions) can improve at least one characteristic (for example taste or smell) of a bean-based product or an intermediate thereof. To enhance the effect of a peroxidase, preferably a Marasmius scorodonius peroxidase or a functional equivalent thereof, an effective amount of hydrogen peroxide can be added.

In one of its aspects, the invention provides a method for improving at least one characteristic of a bean-based product comprising contacting a bean-based product or an intermediate thereof with a peroxidase, preferably a Marasmius scorodonius peroxidase or a functional equivalent thereof, and optionally preparing from said intermediate a bean-based product, wherein said bean-based product or an intermediate is further contacted with hydrogen peroxide, i.e. wherein said bean-based product or an intermediate thereof is contacted with a peroxidase, preferably a Marasmius scorodonius peroxidase or a functional equivalent thereof, and hydrogen peroxide. Contrary to the teaching of EP 0 328 322, in this embodiment of the invention the dissolved oxygen content (in water) is not substantially decreased or eliminated. Preferably, the amount of oxygen is maintained at approximately the same level or is even somewhat increased.

Hydrogen peroxide (H₂O₂) can be obtained in different concentrations and grades/purities. Preferably, the used hydrogen peroxide is food grade. The hydrogen peroxide is added in an effective amount which typically is between 0.1-300 mg H₂O₂ per liter bean-based product or an intermediate thereof. Preferably, the used amount of H₂O₂ is between 0.1-100 mg and even more preferred between 10-60 or between 1-50 and most preferred between 1-10 mg H₂O₂ per liter bean-based product or an intermediate thereof.

The total amount of H₂O₂ can be added in one step. More preferably, the total amount of H₂O₂ is added in more than one step, for example in at least 2, 3, 4 or 5 steps. One can for example add ⅓ of the H₂O₂ at the start of incubation, ⅓ at 15 minutes and the last ⅓ at 30 minutes of incubation. Other dosage schemes can equally well be used.

Surprisingly, some peroxidases can be inactivated if all H₂O₂ is added in one step. The inventors of the current invention have for example determined that the exemplified and preferred peroxidase as described herein, i.e. a Marasmius scorodonius peroxidase or a functional equivalent thereof, can be inactivated by higher concentrations of H₂O₂. In a preferred embodiment, the added amount of H₂O₂ is such that it does not, at least not significantly, inactivate the activity of the used peroxidase. Or the total amount of added H₂O₂ (which total amount would at least partly inactivate the used peroxidase) is added via at least 2, preferably at least 3, more preferably at least 4 separate steps such that the H₂O₂ added in one step does not negatively effect the used peroxidase.

It is furthermore possible to produce the optional H₂O₂ in situ. This can be done by using an oxidoreductase and if the substrate of the used oxidoreductase is not available (or in insufficient amounts) in the bean-based product or an intermediate thereof, by further adding of a substrate for said oxidoreductase.

The used oxidoreductase and the optional substrate for said oxidoreductase are preferably food grade, Halal as well as Kosher.

The invention therefore also provides a method for improving at least one characteristic of a bean-based product comprising contacting a bean-based product or an intermediate thereof with a peroxidase, preferably a Marasmius scorodonius peroxidase or a functional equivalent thereof, and optionally preparing from said intermediate a bean-based product, wherein said bean-based product or an intermediate thereof is contacted with a peroxidase, preferably a Marasmius scorodonius peroxidase or a functional equivalent thereof, and an oxidoreductase.

An oxidoreductase is an enzyme that catalyzes the transfer of electrons from one molecule (the reductant, also called the hydrogen or electron donor) to another (the oxidant, also called the hydrogen or electron acceptor). For example, an enzyme that catalyzed this reaction would be an oxidoreductase:

A⁻+B→A+B⁻

In this example, A is the reductant (electron donor) and B is the oxidant (electron acceptor). According to the proper nomenclature, an x-oxidase uses x as the reductant and O₂ as the acceptor.

The choice for a certain oxidoreductase can be based on the bean-based product or an intermediate thereof. Preferred oxidoreductases are a glucose oxidase, a fructose oxidase and/or a galactose oxidase. Soya contains saccharose (a disaccharide of glucose and fructose), raffinose (a trisaccharide of glucose, fructose and galactose) and stachyose (a tetra saccharide of glucose, galactose and fructose). The enzyme invertase can for example be used to split saccharose into glucose and fructose. After that glucose oxidase and fructose oxidase can be used.

In a preferred embodiment, the used oxidoreductase is a glucose oxidase. In principle each glucose oxidase can be used as long as it displays activity in the bean-based product or an intermediate thereof. The oxidoreductase may be obtained from different sources, including microorganisms. Preferably an oxidoreductase from a fungal source is used, for example from a filamentous fungus. A suitable oxidoreductase is glucose oxidase obtainable from for example an Aspergillus species, such as Aspergillus niger.

As described above, a substrate for the used oxidoreductase can be included as well. In yet another aspect, the invention provides a method for improving at least one characteristic of a bean-based product comprising contacting a bean-based product or an intermediate thereof with a peroxidase, preferably a Marasmius scorodonius peroxidase or a functional equivalent thereof, and optionally preparing from said intermediate a bean-based product, wherein said bean-based product or an intermediate thereof is contacted with a peroxidase (preferably a Marasmius scorodonius peroxidase or a functional equivalent thereof), an oxidoreductase and a substrate for said oxidoreductase.

Examples of suitable combinations of an oxidoreductase and a substrate are: glucose oxidase and glucose (preferably D-glucose). Instead of adding glucose one can also use the sugars which are already present in the corresponding bean and which comprise at least one glucose unit. For example, if the bean is soy, one can use the presence of saccharoase (a disaccharide of glucose+fructose), raffinose (a trisaccharide of glucose+fructose+galactose) or stachyose (tetra saccharide of glucose,+galactose+fructose). For example, invertase can be added to release glucose from saccharose. The glucose oxidase can use the produced glucose as a substrate and produce H₂O₂. The invention thus also provides a method for improving at least one characteristic of a bean-based product comprising contacting a bean-based product or an intermediate thereof with a peroxidase, preferably a Marasmius scorodonius peroxidase or a functional equivalent thereof, and optionally preparing from said intermediate a bean-based product, wherein said bean-based product or an intermediate thereof is contacted with a peroxidase (preferably a Marasmius scorodonius peroxidase or a functional equivalent thereof), an oxidoreductase and an enzyme capable of producing a substrate for said oxidoreductase from a saccharose as already present in the used bean.

In view of costs, a preferred embodiment is the use of a peroxidase, preferably a Marasmius scorodonius peroxidase or a functional equivalent thereof, and H₂O₂.

If H₂O₂ is added or produced in situ, it is preferred that after the contacting (i.e. incubation) phase, the amount of H₂O₂ is consumed by the peroxidase, preferably a Marasmius scorodonius peroxidase or a functional equivalent thereof. This could be important because H₂O₂ is not always allowed in a final product or application. The experimental part describes an experiment in which it is shown that the added amount of H₂O₂ is used by the exemplified and preferred Marasmius scorodonius peroxidase. The invention therefore also provides a method for improving at least one characteristic of a bean-based product comprising contacting a bean-based product or an intermediate thereof with a peroxidase, preferably a Marasmius scorodonius peroxidase or a functional equivalent thereof, and optionally preparing from said intermediate a bean-based product, wherein said bean-based product or an intermediate thereof is contacted with a peroxidase (preferably a Marasmius scorodonius peroxidase or a functional equivalent thereof), and
H₂O₂ or
an oxidoreductase or
an oxidoreductase and a substrate for said oxidoreductase or
an oxidoreductase and an enzyme capable of producing a substrate for said oxidoreductase from a saccharose as already present in the used bean,

wherein the amount of added H₂O₂ or the amount of in situ produced H₂O₂ is chosen/controlled such that after contacting (i.e. incubation with a peroxidase) the amount of H₂O₂ is all used. In such a case, catalase does need to be added for removing any unused H₂O₂. Preferably, a method according to the invention does not use catalase for removing H₂O₂.

Preferably, a method of the invention is performed under conditions which provide oxygen to the reaction mixture. In one its embodiments, the invention provides a method for improving at least one characteristic of a bean-based product comprising contacting a bean-based product or an intermediate thereof with a peroxidase, preferably a Marasmius scorodonius peroxidase or a functional equivalent thereof, and optionally preparing from said intermediate a bean-based product, wherein measures are taken for providing oxygen to the mixture of said bean-based product or an intermediate thereof with said peroxidase (preferably a Marasmius scorodonius peroxidase or a functional equivalent thereof) (optionally with an oxidoreductase with or without a substrate for said oxidoreductase or alternatively an oxidoreductase with an enzyme capable of producing a substrate for said oxidoreductase from a saccharose as already present in the used bean). Oxygen can for example be provided by a magnetic stirring apparatus or by a pump.

In yet another preferred embodiment, the to be treated bean-based product or an intermediate thereof is at least partly liquid or can during the incubation with said peroxidase, preferably a Marasmius scorodonius peroxidase or a functional equivalent thereof, (optionally with an oxidoreductase with or without a substrate for said oxidoreductase) temporarily be made at least partly liquid by for example increasing the temperature or by dissolving in an aqueous medium.

Any bean-based product or an intermediate thereof can be treated by a method according to the invention. Preferably, the term “bean” refers to white beans or soy beans or peas. A method according to the invention is particularly useful on soy-based products or an intermediate thereof, i.e. an intermediate of a soy-based product.

The invention thus provides a method for improving at least one characteristic of a bean-based product comprising contacting a bean-based product or an intermediate thereof with a peroxidase, preferably a Marasmius scorodonius peroxidase or a functional equivalent thereof, and optionally preparing from said intermediate a bean-based product, wherein said bean-based product or an intermediate thereof is a soy-based product or an intermediate thereof.

The term “intermediate thereof” refers to any stage before the bean-based product has obtained its final state (i.e. typically the state in which it is commercially sold). Soy cheese is for example a bean-based product and the soy milk from which the soy cheese is obtained is considered to be an intermediate of said soy cheese. However, soy milk can also be a final product. Herein, reference is also made to a precursor. The terms “precursor” or “intermediate” are used interchangeably herein.

A multitude of soybean-based products exist. Soybeans can be processed to produce a texture and appearance similar to many other foods. In a preferred embodiment, the invention provides a method as described herein, wherein said soy-based product or an intermediate thereof is a dairy substitute such as soy milk, soy cheese, soy drink, soy blend, soy margarine, soy ice cream, soy yoghurt, soy cream cheese or a soy-based dessert.

Alternatively, the soy-based product or an intermediate thereof is a meat substitute. In yet another embodiment, the soy-based product or an intermediate thereof is a fruit drink which comprises soy.

Preferably, the action of the peroxidase, preferably a Marasmius scorodonius peroxidase or a functional equivalent thereof, is directed to a caretonoid. In case of a soy-based product this is for example lutein.

As described above, a method according to the invention results in at least one improved characteristic of a bean-based product or an intermediate thereof. Examples of features (or characteristics; the terms are used interchangeably herein) are taste, smell, mouthfeel, colour (whiteness) and viscosity. In case the bean-based product or an intermediate thereof is a soybean-based product or an intermediate thereof features like soy bean taste and soy bean smell can be reduced. More in specific it is possible to improve (i.e. reduce) the intensity smell, the intensity taste, off-flavour (such as medical taste and/or sulphur taste) can be improved. In respect of a soybean-based product and viscosity it is noted that the viscosity is decreased when a method according to the invention is applied, i.e. the product is considered to be less sticky.

The invention also provides a method for improving at least one of taste, smell, mouthfeel, colour (whiteness) and viscosity of a bean-based product comprising contacting a bean-based product or an intermediate thereof with a peroxidase, preferably a Marasmius scorodonius peroxidase or a functional equivalent thereof, and optionally preparing from said intermediate a bean-based product. In one of its preferred embodiments, the to be improved feature is colour, i.e. a more white colour. The invention therefore also provides a method for improving the colour (whiteness) of a bean-based product (alternatively, a method for producing a bean-based product with a more white colour) comprising contacting a bean-based product or an intermediate thereof with a peroxidase, preferably a Marasmius scorodonius peroxidase or a functional equivalent thereof, and optionally preparing from said intermediate a bean-based product.

Based on the characteristic which must be improved, different methods for determining whether the characteristic indeed has improved can be applied.

The color can for example be determined in time with a X-rite 968 reflections spectrophotometer where 3 components are determined: L=black/white, a=green/red, b=yellow/blue. For soy milk, component b is most changeable and sensitive for yellow color of soy milk. This value can thus be used to follow the color.

In respect of taste and smell two types of panels are used. The first panel consisted of experts that are selected on their ability to taste and smell very well and they are trained for sensory analyses and use the same vocabulary to describe products. This panel is sensitive for small differences between samples. But for products in the supermarket it is important that the consumers can taste the improvement of a treated product. In this respect it is normal to use a non selected non trained panel of consumers, and to apply a triangle test method to determine whether or not there is a difference in taste and smell between a treated and a non treated product. Each panelist gets 3 samples of which 2 are equal and 1 is different. For instance a panelist gets 2 treated products which are duplicates, and 1 non treated product. But it can also be 1 treated and 2 non treated samples. All 3 samples have a different code. The codes and the sequence differ per panelist, and are only known to the panel-leader. Each panelist has to determine the odd one. The chance to guess right is 1 out of 3, so 33% of the results are correct by chance, by guessing. Depending on the amount of panelists one can calculate how big the % of right answers should be to conclude that there is a significant difference between the treated and non treated product. To increase the certainty the panel should consist of enough people.

The invention provides a method for improving at least one characteristic of a bean-based product comprising contacting a bean-based product or an intermediate thereof with a peroxidase, preferably a Marasmius scorodonius peroxidase or a functional equivalent thereof, and optionally preparing from said intermediate a bean-based product. Preferably, said at least one characteristic is smell or taste. Even more preferably at least two characteristics are improved, such as taste and smell. Even more preferably, at least three, four or 5 characteristics are improved.

The invention therefore also provides a method for improving at least one characteristic of a bean-based product comprising contacting a bean-based product or an intermediate thereof with a peroxidase, preferably a Marasmius scorodonius peroxidase or a functional equivalent thereof, and optionally preparing of said intermediate a bean-based product, wherein at least two characteristics of a bean-based product are improved. Preferably, said at least two characteristics are (i) colour (whiteness) and taste or (ii) taste and smell or (iii) colour (whiteness) and viscosity or (iv) colour (whiteness) and smell.

The used peroxidase can be any type of peroxidase.

Peroxidases (EC number 1.11.1.x) are a large family of enzymes. A majority of peroxidase protein sequences can be found in the PeroxiBase database. Peroxidases typically catalyze a reaction of the form:

ROOR′+electron donor (2 e⁻)+2H⁺→ROH+R′OH

For many of these enzymes the optimal substrate is hydrogen peroxide, but others are more active with organic hydroperoxides such as lipid peroxides. Peroxidases can contain a heme cofactor in their active sites, or redox-active cysteine or selenocysteine residues.

The classification and nomenclature of enzymes is not always directly and unambiguously clear. More specific, it is not clear whether or not catalase is a real peroxidase. According to the website http://www.chemamu.gmul.ac.uk/iubmb/enzyme/rules.html: “Peroxidase is used for enzymes using H₂O₂ as acceptor. Catalase must be regarded as exceptional”. Using the website: http://us.expasy.org/cgi-bin/enzyme-search-de, it is again clear that catalase behaves differently, because catalase is not mentioned in a peroxidase search. In other enzyme lists, catalase is mentioned as EC 1.11.1.6 which is missing in the latter website when searching for peroxidase. To avoid any misunderstanding, the peroxidase as used in a method or product as claimed herein does not include a catalase, i.e. in a preferred embodiment, the invention provides a method for improving at least one characteristic of a bean-based product comprising contacting a bean-based product or an intermediate thereof with a peroxidase and optionally preparing of said intermediate a bean-based product, with the proviso that said peroxidase is not a catalase.

In one its embodiments, the preferred peroxidase is a beta-carotene converting enzyme and even more preferably a lutein converting enzyme. The invention therefore provides a method for improving at least one characteristic of a bean-based product comprising contacting a bean-based product or an intermediate thereof with a peroxidase, preferably a Marasmius scorodonius peroxidase or a functional equivalent thereof, and optionally preparing from said intermediate a bean-based product, wherein said peroxidase is a beta-carotene converting enzyme.

An example of a beta-carotene converting enzyme is the soy enzyme lipoxygenase. Another example is lactoperoxidase. In a preferred embodiment, said beta-carotene converting enzyme is a Marasmius, for example Marasmius scorodonius, beta-carotene converting enzyme, i.e. an enzyme naturally expressed by Marasmius. WO 2007/006792 describes multiple Marasmius beta-carotene converting enzymes, i.e. caroase 01-05. Caroase 01, 02, 03, 04 or 05 are also referred to as MsP1, MsP2, MsP3, MsP4 or MsP5 respectively (the term caroase and MsP are used interchangeably herein). Even more preferred, the used enzyme is caroase 01 or 02, i.e. MsP1 or MsP2.

Most preferred is the use of a protein which is encoded by nucleotide sequence SEQ ID NO: 4 of WO 2007/006792 (as used herein within the experimental part) or a protein encoded by another nucleotide sequence but which another nucleotide sequence results in a protein identical to the one encoded by the nucleotide sequence of SEQ ID NO:4 of WO 2007/006792, e.g. a nucleotide sequence which has alternative Wobble position nucleotides or a protein encoded by a nucleotide sequence having at least about 55%, 60%, 65%, 75%, 80%, 85%, 90%, 95%, 96%, 97%, 98% or 99% identity with SEQ ID NO:4 of WO 2007/006792.

The caroase-01-05 bleaching enzymes suitable for use in a method according to the invention, or functional equivalents thereof, can be recovered and purified from (recombinant) cell cultures by well-known methods including ammonium sulphate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

The caroase-01-05 bleaching enzymes suitable for use in a method according to the invention, include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, the caroase-01-05 bleaching enzymes may also include an initial modified methionine residue, in some cases as a result of host-mediated processes.

A functional equivalent of a caroase 01-05 polypeptide (or alternatively a functional equivalent of a Marasmius scorodomius peroxidase) is a polypeptide which activity on a bean-based product or an intermediate thereof results in at least one improved characteristic of a bean-based product, for example a polypeptide that exhibits at least the same or a better bleaching activity or at least the same or a better taste or smell of at least one of the caroase 01-05 Marasmius scorodonius bleaching enzymes as described herein. Such a functional equivalent can equally well be used in a method of the invention. Preferably, a functional equivalent of a Marasmius scorodomius peroxidase has at least about 55%, 60%, 65%, 75%, 80%, 85%, 90%, 95%, 96%, 97%, 98% or 99% identity with the protein as encoded SEQ ID NO: 4 of WO 2007/006792 and exhibits at least the same or a better bleaching activity or at least the same or a better taste or smell when compared to the Marasmius scorodonius peroxidase as exemplified herein.

Functional protein or polypeptide equivalents may contain only conservative substitutions of one or more amino acids of any one of SEQ ID NO: 08-12 (as published with the WO 2007/006792 publication) or substitutions, insertions or deletions of non-essential amino acids of any of these. Accordingly, a non-essential amino acid is a residue that can be altered in any one of said SEQ ID NO: 08-12 (preferably SEQ ID NO: 9-11, i.e. SEQ ID No: 9 or 10 or 11) without substantially altering the biological function. For example, amino acid residues that are conserved among the caroase 01-05 proteins of are predicted to be particularly unamenable to alteration. Furthermore, amino acids conserved among the caroase 01-05 proteins and other peroxidases are not likely to be amenable to alteration.

The term “conservative substitution” is intended to mean that a substitution in which the amino acid residue is replaced with an amino acid residue having a similar side chain. These families are known in the art and include amino acids with basic side chains (e.g., lysine, arginine and hystidine), acidic side chains (e.g. aspartic acid, glutamic acid), uncharged polar side chains (e.g., glycine, asparagines, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine tryptophan, histidine).

Caroase 01-05 proteins that contain changes in amino acid residues that are not essential for a particular biological activity. Such caroase 01-05 proteins differ in amino acid sequence from any one of SEQ ID NO: 08-12 of WO 2007/006792 yet retain at least one biological activity. In one embodiment the isolated nucleic acid molecule comprises a nucleotide sequence encoding a protein, wherein the protein comprises a substantially homologous amino acid sequence of at least about 55%, 65%, 70%, 75%, 80%, 85%, 90%, 95%, 96%, 97%, 98%, 99% or more homologous to the amino acid sequence shown in any one of SEQ ID NO: 08-12 as published with the WO 2007/006792 publication.

For example, guidance concerning how to make phenotypically silent amino acid substitutions is provided in Bowie, J. U. et al., Science 247:1306-1310 (1990) wherein the authors indicate that there are two main approaches for studying the tolerance of an amino acid sequence to change. The first method relies on the process of evolution, in which mutations are either accepted or rejected by natural selection. The second approach uses genetic engineering to introduce amino acid changes at specific positions of a cloned gene and selects or screens to identify sequences that maintain functionality. As the authors state, these studies have revealed that proteins are surprisingly tolerant of amino acid substitutions. The authors further indicate which changes are likely to be permissive at a certain position of the protein. For example, most buried amino acid residues require non-polar side chains, whereas few features of surface side chains are generally conserved. Other such phenotypically silent substitutions are described in Bowie et al, supra, and the references cited therein.

The term “functional equivalents” also encompasses orthologues of the M. scorodonius caroase 01-05 protein. Orthologues of the M. scorodonius caroase 01-05 protein are proteins that can be isolated from other strains or species and possess a similar or identical biological activity. Such orthologues can readily be identified as comprising an amino acid sequence that is substantially homologous to any one of SEQ ID NO: 08-12 of WO 2007/006792.

In a preferred embodiment, the invention provides a method for improving at least one characteristic of a bean-based product comprising contacting a bean-based product or an intermediate thereof with a peroxidase and optionally preparing of said intermediate a bean-based product, wherein said peroxidase is a Marasmius scorodonis peroxidase.

The enzymes (i.e. a peroxidase and optionally an oxidoreductase) are added in effective amounts. The skilled person can easily determine this effective amount by varying the enzyme dosage and determining any of the herein described characteristics (such as smell, taste or color). In case the enzymes are capable of converting beta-carotene, the effective amount of enzymes may be expressed in terms of beta-degrading units, e.g. Aziz or Zorn units.

One or multiple of the used enzyme(s) may be added as an enzyme preparation or produced in situ by a microorganism capable of producing a particular (combination of) enzyme(s). An enzyme preparation can be derived from various sources, for example from plants, animals and microorganisms. Preferably an enzyme preparation is derived from a microorganism, since microorganisms make it possible to obtain the enzyme on an industrial scale in a controlled manner. An enzyme preparation derived from a microorganism can be obtained by classical fermentation processes of a selected microbial strain or by fermentation of a microorganism that over expresses the enzyme. The microorganism may be a bacterium, a fungus or yeast. Examples of suitable microorganisms are Microcystis, Lepista, for example L. irina, Cyathus, for example C. pallidus, Ganoderma, for example G. applanatum, Ischnoderma, for example I. benzoinum, Marasmius, for example M. scorodonius, Trametes, for example T. suaveoluens of T. versicolour, Cryptococcus, for example C. laurentii, Hypomyces, for example H. odoratus or Phaffia, for example P. rhodozyma, Phanerochaete for example P. chrysosporium, Lentinula for example L. edodes, Coprinus for example C. cinereus, Gloeophyllum for example G. trabeum, Ophiostoma for example O. piliferum, Aspergillus for example A. niger, A. oryzae, A. nidulans, Thermomyces, for example T. lanuginosa, Sporotrichum, for example S. thermophile, Aureobasidium for example A. pullulans, Amorphotheca, for example A. resinae, Leucosporidium, for example L. scottii, Cunninghamella, for example C. elegans.

Especially preferred are cells from filamentous fungi, in particular Aspergillus—for example Aspergillus oryzae or Aspergillus niger—and Marasmius—for example Marasmius scorodonius—or cells from yeasts such as Pichia,—for example Pichia Pastoris—or cells from bacteria.

Host cells also include, but are not limited to, mammalian cell lines such as CHO, VERO, BHK, HeLa, COS, MDCK, 293, 3T3, WI38, and choroid plexus cell lines.

A method according to the invention can be complemented by adding at the end of the incubation an effective amount of a catalase to inactivate any remaining H₂O₂ activity although it is preferred that the amount of added (at once or in phases) H₂O₂ is selected such that inactivation by catalase is not necessary (see above).

A method according to the invention can further comprise a final treatment (for example a heat treatment) to inactivate the used enzyme(s), i.e. the used peroxidase and optionally the used oxidoreductase and/or the used catalase. The invention therefore also provides, a method for improving at least one characteristic of a bean-based product comprising contacting a bean-based product or an intermediate thereof with a peroxidase, preferably a Marasmius scorodonius peroxidase or a functional equivalent thereof, and optionally preparing from said intermediate a bean-based product, further comprising inactivating said peroxidase and/or optionally said oxidoreductase and/or optionally said catalase.

Preferably a method according to the invention is performed on a large scale, i.e. in large volumes, for example starting from 1 liter and extending to 100000 liters. Even more preferably a method according to the invention is performed in a volume of at least 2, 3, 4, 5, 6, 7, 8, 9, or 10 liters.

There are a large amount of different recipes for producing soy milk and in principle a method according to the invention can be incorporated in any such process or said processes can be amended slightly without any effort to include the method of the invention.

Soy milk can be made from whole soybeans or full-fat soy flour. The dry beans are typically soaked in water overnight or for a minimum of 3 hours or more depending on the temperature of the water. The rehydrated beans then undergo wet grinding with enough added water to give the desired solids content to the final product. The ratio of water to beans on a weight basis should be about 10:1. The resulting slurry or purée is brought to a boil in order to improve its nutritional value by heat inactivating soybean trypsin inhibitor, improve its flavor and to sterilize the product. Heating at or near the boiling point is continued for a period of time, 15-20 minutes, followed by the removal of an insoluble residue (soy pulp fiber or okara) by filtration.

There is a simple yet profound difference between traditional Chinese and Japanese soy milk processing: the Chinese method boils the filtrate (soy milk) after a cold filtration, while the Japanese method boils the slurry first, followed by hot filtration of the slurry. The latter method results in a higher yield of soy milk but requires the use of an anti-foaming agent or natural defoamer during the boiling step. Bringing filtered soy milk to a boil avoids the problem of foaming. It is generally opaque, white or off-white in color, and approximately the same consistency as cow's milk.

In one of its aspects, the invention provides a method for improving at least one characteristic of a bean-based product comprising contacting a bean-based product or an intermediate thereof with a peroxidase, preferably a Marasmius scorodonius peroxidase or a functional equivalent thereof, and optionally preparing from said intermediate a bean-based product, wherein said peroxidase is contacted with a bean-based product or an intermediate thereof after a bean soaking step, for example by adding said peroxidase after grinding, e.g. just after grinding or by incubating prepared soy milk with said peroxidase.

In yet another aspect, the invention provides a method for improving at least one characteristic of a bean-based product comprising contacting a bean-based product or an intermediate thereof with a peroxidase, preferably a Marasmius scorodonius peroxidase or a functional equivalent thereof, and optionally preparing of said intermediate a bean-based product, wherein said peroxidase is contacted with a bean-based product or an intermediate thereof during a bean soaking step. As described above, soaking is typically performed in water overnight or for a minimum of 3 hours. During this soaking phase there is ample time for said peroxidase to improve at least one characteristic of a bean-based product.

The invention further provides a method which combines the latter two described incubations, i.e. a method for improving at least one characteristic of a bean-based product comprising contacting a bean-based product or an intermediate thereof with a peroxidase, preferably a Marasmius scorodonius peroxidase or a functional equivalent thereof, and optionally preparing of said intermediate a bean-based product, wherein said peroxidase is contacted with a bean-based product or an intermediate thereof during a bean soaking step and wherein the peroxidase treated bean-based product or an intermediate thereof is further contacted with a peroxidase after grinding (i.e. post grinding). As will be exemplified later on, such a dual peroxidase treatment results in more white colour of a bean-based product when compared to treatment during soaking only or post grinding only.

In yet another preferred embodiment, the beans treated in a method according to the invention are pretreated to remove the hulls from the beans prior to a soaking step. The invention therefore also provides a method for improving at least one characteristic of a bean-based product comprising contacting a bean-based product or an intermediate thereof with a peroxidase, preferably a Marasmius scorodonius peroxidase or a functional equivalent thereof, and optionally preparing of said intermediate a bean-based product, wherein the hulls of said beans are removed before subjecting the beans to a Marasmius scorodonius peroxidase or a functional equivalent thereof. This results in a further improved bean-based product.

In yet a further embodiment, the invention provides a method for improving at least one characteristic of a bean-based product comprising contacting a bean-based product or an intermediate thereof with a peroxidase, preferably a Marasmius scorodonius peroxidase or a functional equivalent thereof, and optionally preparing of said intermediate a bean-based product, wherein the amount of reducing sugars is essentially not changed, i.e. a method in which the amount of reducing sugars is not increased when compared to the amount of reducing sugars before peroxidase treatment. The improvement in at least one characteristic of a bean-based product is not obtained by an increase in the amount of reducing sugars and the subsequent reaction of reducing sugars with bean proteins to produce flavors which are capable of masking certain objectionable flavors in a bean-based product. The obtained improvements in a method according to the invention are (solely) due to the action of the peroxidase (optionally in combination with H₂O₂ or an oxidoreductase and optionally a substrate for said oxidoreductase).

As described above as well as in the experimental part, the product obtainable by a method according to the invention has improved characteristics.

The invention therefore also provides a bean-based product (for example a soybean-based product) obtainable according to any one of the herein described methods. Such a product typically has improved (i.e. whiter) color or taste less like beans or smells less like beans.

The invention further provides use of a peroxidase, preferably a Marasmius scorodonius peroxidase or a functional equivalent thereof, to improve at least one characteristic of a soy-based product or an intermediate thereof.

The used peroxidase, preferably a Marasmius scorodonius peroxidase or a functional equivalent thereof, and optionally the used oxidoreductase, the used substrate for said oxidoreductase and the optional catalase (to inactivate any remaining H₂O₂) and an enzyme capable of producing a substrate for said oxidoreductase from a saccharose as already present in the used bean can be added via a liquid or a solid formulation. Liquid compositions are preferred due to the fact that they can be pumped.

If a method according to the invention uses a peroxidase, preferably a Marasmius scorodonius peroxidase or a functional equivalent thereof, as well as an oxidoreductase the enzymes can be added at the same time or within a short time period from each other. For example, one first adds said oxidoreductase and after a couple of minutes the peroxidase is added as well or vice versa. If a method according to the invention applies a peroxidase, preferably a Marasmius scorodonius peroxidase or a functional equivalent thereof, and hydrogen peroxide, these components can as well be added in any desired order. As already mentioned, the hydrogen peroxide can be added at once or in phases at different time points.

To improve the ease of handling and as a consequence to reduce the risk of mistakes, the components peroxidase (preferably a Marasmius scorodonius peroxidase or a functional equivalent thereof), oxidoreductase and a substrate for said oxidoreductase are provided in one unit. Preferably, said one unit has at least two separate compartments and the contents of said two compartments can be mixed by removing a barrier between the two compartments. An example of such a unit is a dual chamber package. Preferably, the oxidoreductase and its substrate are separated from each other. The invention thus provides a dual chamber package comprising in a first chamber a combination of a peroxidase, preferably a Marasmius scorodonius peroxidase or a functional equivalent thereof, and an oxidoreductase and in a second chamber a substrate for said oxidoreductase or in a first chamber a combination of peroxidase, preferably a Marasmius scorodonius peroxidase or a functional equivalent thereof, and a substrate for an oxidoreductase and in a second chamber an oxidoreductase.

However, if suitable measures are taken said peroxidase (preferably a Marasmius scorodonius peroxidase or a functional equivalent thereof), oxidoreductase and the substrate for the oxidoreductase can be present in one and the same storage or packaging compartment. The inventors of the present invention noted that upon removal of oxygen the mentioned components can be added together. Hence, the invention provides a vacuum packed composition of a peroxidase (preferably a Marasmius scorodonius peroxidase or a functional equivalent thereof), an oxidoreductase and a substrate for said oxidoreductase. The inventors further noticed that upon reducing the water activity a stable composition of said peroxidase (preferably a Marasmius scorodonius peroxidase or a functional equivalent thereof), oxidoreductase and said substrate can be prepared as well. Preferably, the water activity is reduced by adding glycerol, preferably in a concentration of at least 50, 60, 70, 80 or 90%. The invention thus also provides a composition comprising a peroxidase (preferably a Marasmius scorodonius peroxidase or a functional equivalent thereof), an oxidoreductase and a substrate for said oxidoreductase which composition has a reduced water activity.

In yet another embodiment, the invention provides a kit comprising a peroxidase (preferably a Marasmius scorodonius peroxidase or a functional equivalent thereof), an oxidoreductase and optionally a substrate for said oxidoreductase. Such a kit can comprise separate bottles or tubes for the different components or 2 of said components can be premixed, for example a peroxidase (preferably a Marasmius scorodonius peroxidase or a functional equivalent thereof) and an oxidoreductase or a peroxidase and a substrate for an oxidoreductase. The invention further provides a kit comprising a peroxidase (preferably a Marasmius scorodonius peroxidase or a functional equivalent thereof) and H₂O₂.

Finally, the peroxidase (preferably a Marasmius scorodonius peroxidase or a functional equivalent thereof) used in a method according to the invention, optionally in combination with an oxidoreductase and an optional substrate can be presented as a solid (dried) composition.

A method according to the invention typically requires 0, 1-300, preferably 5-100, more preferably 10-50 or 1-50 and even more preferably 1-10 mg H₂O₂ per liter bean-based product. Typical ratios used in a method according to the invention as well as in the above described compositions are between 0.05-5 gram substrate/liter and between 0.1-18 units of an oxidoreductase and 0.1 to 10 units of peroxidase. The mentioned ranges are especially applicable for the combination of a Marasmius peroxidase, glucose oxidase as an oxidoreductase and glucose as a substrate for said glucose oxidase.

The invention will be explained in more detail in the following examples, which are not limiting the invention.

### Example 1

Bleaching experiments were performed in fresh intermediate soy milk product.

The composition of the product was:

Protein: 5.59%

Dry matter: 10.5%

pH: 6.9

The soy milk bleaching was performed on 1 L scale and experimental conditions were:

T=35° C., mixing using magnetic stirring at 100 rpm. In order to prevent inactivation of Msp1 (Marasmius scorodonius protein 1) by to high concentration of H₂O₂ the H₂O₂ was added in five steps at t=0, t=10 min, t=20 min, t=30 min. and t=60 min.

Sample Codes:

1. H₂O₂ (final concentration 1 mmol/l)

2. MsP1 dose 2.25 DBLU/ml+H₂O₂ (final concentration 1 mmol/l)

3. MsP1 dose 4.5 DBLU/ml+H₂O₂ (final concentration 1 mmol/l)

4. MsP1 dose 9 DBLU/ml+H₂O₂ (final concentration 1 mmol/l)

5. MsP1 dose 9 DBLU/ml without H₂O₂

The MsP activity is expressed in DBLU (Dairy BLeaching Units). One DBLU is defined as the amount of enzyme that oxidizes 1 μmol ABTS per minute under conditions of the test (37° C., pH 3.50)

MsP catalyses the reduction of hydrogen peroxide at pH 3.50 and at 37° C., while oxidizing 2,2′ azino-bis-3-ethylbenzthiazoline-6-sulfonic acid (ABTS) forming a green coloured complex that can be measured spectrophotometrically at 405 nm. The method is for example performed with the Konelab analyser.

The color of soy milk was followed in time visually by making photos and measured by means of X-rite 968 reflection spectrophotometer where the 3 components are determined: L=black/white, a=green/red, b=yellow/blue. It appeared that component b is most changeable and sensitive for yellow color of soy milk and therefore was used to determine of bleaching efficiency.

Results

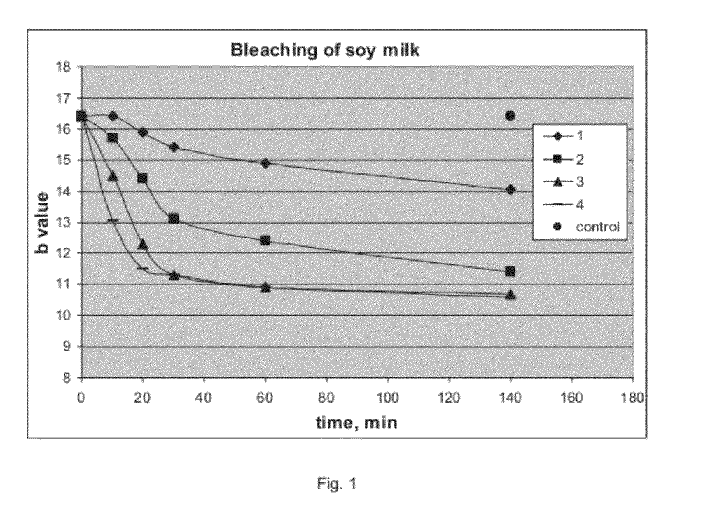

The results of soy milk bleaching are presented in FIG. 1 where b component of color was measured in time.

### Example 2

The soy milk bleaching was performed on 200 ml scale and experimental conditions were following: T=21° C. and T=35° C., mixing using magnetic stirring at 200 rpm.

MsP1 was food grade material (batch DBL.GRZ.0848.58).

GOX was food grade (GO10 000 batch 408201701).

Glucose was D-glucose Sigma ultra batch 075K0022.

Two blends of MsP1+GOX+glucose were tested that were previously optimized for another bleaching application.

TABLE 1
Changes in b component of soy milk color during bleaching process.

| | Glucose | GOX | MsP1 |
|---|---|---|---|
| | (g/L) | (SRU/ml) | (DBLU/ml) |
| Blend 1 | 0.2 | 0.18 | 1.25 |
| Blend 2 | 0.81 | 0.18 | 2.25 |

Glucose oxidase activity assays are well known. One non-limiting example is described. Glucose oxidase catalyses the oxidation of glucose into gluconic acid at pH 5.40 and at 37° C. releasing hydrogen peroxide. Under the influence of peroxidase, the hydrogen peroxide is reduced to water while oxidizing 2,2′ azino-bis-3-ethylbenzthiazoline-6-sulfonic acid (ABTS) forming a green coloured complex that can be measured spectrophotometrically at 405 nm. The method is for example performed with the Konelab analyser. This is a relative method. The results are related to a glucose oxidase preparation with an officially assigned activity.

The activity is expressed in SRU (Sarrett Units). One SRU unit is defined as the amount of enzyme that gives an oxygen uptake of 10 m³/minute in a Warburg manometer, at 30° C. and in the presence of an excess of oxygen, catalase and a 3.3% glucose solution in a phosphate buffer of pH 5.9.

For samples blend 1 and blend 2 were used and an aquarium pump was used to enrich soy milk with oxygen.

In order to prevent inactivation of Msp1 by to high concentration of H₂O₂ the H₂O₂ was added in three steps at t=0, t=15 min, t=30 min to the final concentration of H₂O₂ 0.9 mmol/l. Using two enzyme system and glucose, first glucose was added than Msp1 enzyme and as last GOX

Sample Codes:

1. T=21° C., Blend 1

2. T=35° C., Blend 1

3. T=21° C., Blend 2

4. T=35° C., Blend 2

5. T=21° C., MsP1 dose 2.25 DBLU/ml+H₂O₂

6. T=35° C., MsP1 dose 2.25 DBLU/ml+H₂O₂

7. T=21° C., MsP1 dose 4.5 DBLU/ml+H₂O₂

8. T=35° C., MsP1 dose 4.5 DBLU/ml+H₂O₂

9. T=35° C. MsP1 dose 4.5 DBLU/ml

10. T=35° C., H₂O₂ (final concentration 0.9 mmol/l)

11. T=35° C., glucose 0.8 g/l+GOX 0.18SRU/ml

12. T=35° C., untreated soy milk

The color of soy milk was followed in time by means of X-rite 968 reflection spectrophotometer where the 3 components are determined: L=black/white, a=green/red, b=yellow/blue. It appeared that component b is most changeable and sensitive for yellow color of soy milk and therefore was used to monitor the bleaching efficiency.

Since soy milk was intermediate and not heat treated product after ˜150 min incubation at T=35° C. and T=21° C. samples were placed in cool room at T=8° C. and finally color was analyzed after ˜1000 min.

Results

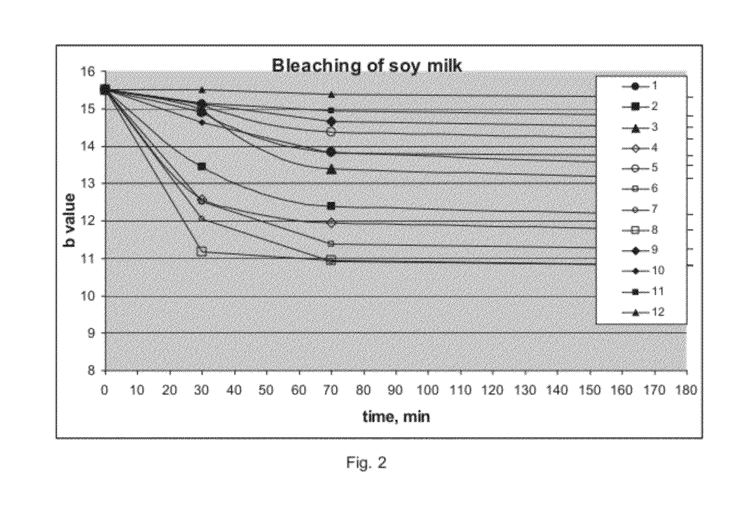

The results of soy milk bleaching are presented in FIG. 2 and in Table 2 where b component of color was measured in time.

TABLE 2
Changes in b component of soy milk color during bleaching process.

| | 1 | 2 | 3 | 4 | 5 | 6 | 7 | 8 | 9 | 10 | 11 | 12 |
|---|---|---|---|---|---|---|---|---|---|---|---|---|
| | 21° C. | 35° C. | 21° C. | 35° C. | 21° C. | 35° C. | 21° C. | 35° C. | 35° C. | 35° C. | 35° C. | 35° C. |
| t, min | Blend1 | Blend1 | Blend2 | Blend2 | dose1 | dose1 | dose2 | dose2 | dose2 | H₂O₂ | GOX + Glu | control |
| 0 | 15.5 | 15.5 | 15.5 | 15.5 | 15.5 | 15.5 | 15.5 | 15.5 | 15.5 | 15.5 | 15.5 | 15.5 |
| 30 | 14.9 | 13.4 | 15.0 | 12.6 | 15.1 | 12.5 | 12.1 | 11.2 | 15.1 | 14.6 | 15.2 | 15.5 |
| 70 | 13.8 | 12.4 | 13.4 | 12.0 | 14.4 | 11.4 | 10.9 | 10.9 | 14.7 | 13.8 | 14.9 | 15.4 |
| 1000* | 11.4 | 11.0 | 12.0 | 10.6 | 13.2 | 10.3 | 10.1 | 9.9 | 13.4 | 13.3 | 13.8 | 14.8 |

*The b values at time 1000 min were measured for samples that were stored at T = 8° C.

As can be seen from FIG. 2 and Table 2 the color of soy milk was mostly changed during first 100 min of incubation and than did not change significantly. However it has to be noticed that after ˜150 min of incubation samples were placed in cool room at T=8° C. and during storage no H₂O₂ was added.

The best bleaching was achieved when combination of MsP1+H₂O₂ (samples 6, 7, 8) was used and moreover at higher dose of MsP1 (samples 7, 8). Sample 5 was out of expected bleaching range that probably due to some mistake during sample preparation. The final bleaching efficiency was not temperature dependent but most likely bleaching is faster at T=35° C. during first 30 minutes.
The bleaching of soy milk when blend Msp1+GOX+glucose used (samples 1,2,3,4) was less effective compare to Msp1+H₂O₂ that could due to the oxygen shortage. During bleaching the oxygen (air) was blown using aquarium pump but this was not continuous process since this gives foam forming in soy milk.
The bleaching efficiency using blends is temperature dependent if samples 1 with 2 and 3 with 4 are compared and significantly higher at higher temperature. The concentrations of Msp1 and glucose in blend1 and in blend2 have not significant effect on bleaching efficiency samples 1, 3 and sample 2, 4. Although probably bleaching efficiency is slightly higher at higher concentrations of MsP1 and glucose.
The “sniffing” test of samples 1-12 revealed slight differences in odor between reference soy milk, sample 12 and treated samples 1-8. The difference was small and additional tests with trained panel are required to make a final conclusion.

### Example 3

For taste experiments the soy milk bleaching was performed on 500 ml scale and experimental conditions were following: T=35° C., mixing using magnetic stirring at 200 rpm.

MsP1 was food grade material (batch DBL.GRZ.0848.58).

GOX was food grade (batch GO10 000 batch 408201701).

Glucose was D-glucose Sigma ultra batch 075K0022.

Bleaching experiments were performed in fresh intermediate soy milk product.

The composition of the product was:

Protein: 5.59%

Dry matter: 10.5%

pH: 6.9

Three soymilk treatments with MSP1 were tested on taste.

Using two enzyme system and glucose, first glucose was added than Msp1 enzyme and as last GOX. To enrich soy milk with oxygen an aquarium pump was used.

In order to prevent inactivation of Msp1 (Marasmius scorodonius protein 1) by to high concentration of H₂O₂ the H₂O₂ was added in five steps at t=0, t=10 min, t=20 min, t=30 min. and t=60 min.

Sample Codes:

1. MSP1 dose 2.25 DBLU/ml+glucose oxidase 180 units/l+glucose 0.8 gram/l

2. MsP1 dose 4.5 DBLU/ml+H₂O₂ (final concentration 1 mmol/l)

3. MsP1 dose 4.5 DBLU/ml

Control

To prepare food grade samples the samples were pasteurized during 10 minutes at 85° C. in a waterbath. After cooling down the treated samples and the control sample to room temperature the samples were tasted by an untrained taste panel of 24 people. Next to several attributed such as soybean taste, grass/green/flower taste and off flavor, intensity of taste/smell and preference were tested.

Results

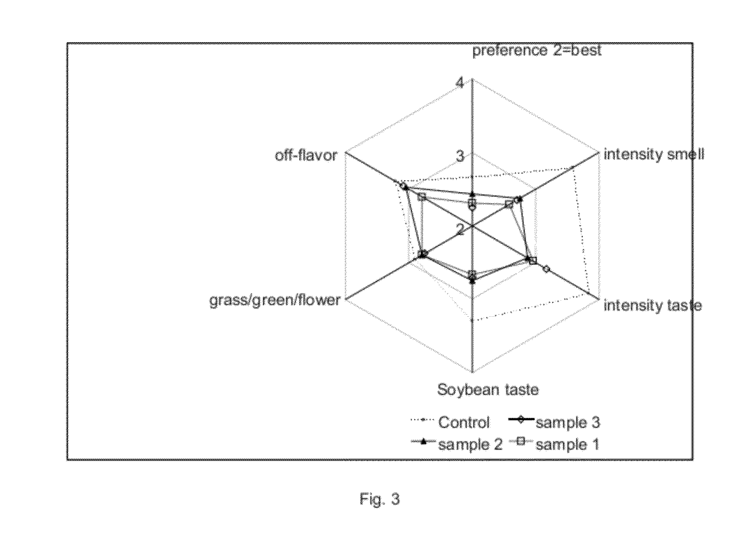

As can be seen from FIG. 3 the intensity of smell and taste were less than the control sample. Also taste of soybean taste, grass/green/flower taste and off flavor changed significantly. The taste panel preferred the taste of all three samples with MsP1. The outcome of all treated samples was comparable.

### Example 4

### Sensory Triangle Test

### Summary

The purpose is to test and investigate whether there is a difference or not.

An adequate amount of assessors will be targeted in order to achieve sufficient protection against the type-II error (i.e. the risk of falsely accept HO “no difference” where there is a true error).

Samples

Suitable samples are:

### Concept 1.

Ingredients per liter Soy milk

MSP1 with H₂O₂ Generating System.

Glucose: 0.8 g glucose

GOX: 180 U/I (18 mg Bakezym 10000/l)

MSP1: 2250 DBLU/I (0.34 ml filtrate (6554 DBLU/ml)

Aerate soy milk using an aquarium pump

Incubation temperature: 35° C.

Incubation time: 1 hour

### Concept 2.

Ingredients per liter Soy milk

MSP1 with H₂O₂ Added.

MSP1: 2250 DBLU/I (0.34 ml filtrate (6554 DBLU/ml))

Starting at T=0 minutes; add during one hour every 15 minutes 0.66 ml 1% H₂O₂ solution (in total 5×6.6 mg H₂O₂/I soy milk)

Incubation temperature: 35° C.

Incubation time: 1 hour

### Concept 3.

Ingredients per liter Soy milk

MSP1 without Bleaching.

MSP1: 2250 DBLU/I (0.34 ml filtrate (6554 DBLU/ml)

Incubation temperature: 35° C.

Incubation time: 1 hour.

### Concept 4.

Reference: like concept 3 but without Msp1.

After incubation the treated soy milk is pasteurized in a water bath during 10 minutes at 85° C. After pasteurization the soy milk is cooled down in a water bath of 4° C. For tasting the samples are served at room temperature.

Sample Preparation and Serving

The samples will be served at room temperature in plastic cups. The samples are judged by a sufficient amount of people. Each assessor evaluates one triangle test.

Data Acquisition

The data acquisition is done by use of paper answer form.

Data Analysis

In a triangle test, three samples are presented simultaneously to the panelists; two samples are from the same formulation and one is from the different formulation.

Each panelist has to indicate the odd sample. There are six possible serving orders (AAB, ABA, BAA, BBA, BAB, ABB) that should be counterbalanced across all panelists.

Power computations and statistical inferences about the percentage correctly classified odds will be made according to the methods published by Schlich et al. (1993) and Brockhoff (2003).

Results

The null hypothesis for the triangle test states that the probability of making a correct selection when there is no perceptible difference between the samples is one in three.

### Example 4

### Effect Start H₂O₂ Concentration on Bleaching Activity of Marasmius Scorodonius Peroxidase and H₂O₂ Residues in Soymilk Application

In order to test bleaching capacity of Marasmius scorodonius peroxidase and H₂O₂ residues in soymilk after bleaching, the effect of several H₂O₂ concentrations was tested under set conditions.

Tests were done in a soy milk intermediate product. The composition of the product was following:

Protein: 5.59%

Dry matter: 10.5%

pH: 6.9

Experiments were executed in dark environment at a temperature of 7° C. Marasmius scorodonius peroxidase was tested in a concentration of 2.25 DBLU Marasmius scorodonius peroxidase/ml. whey. Tests were done with 0.18 mM H₂O₂ added to the soymilk.

Results:
In 5 hours, the b-value decreased from 15.3 to 11.7.
Under the tested (optimal) conditions (0.18 mM H₂O₂) all H₂O₂ is used by Marasmius scorodonius peroxidase within 5 hours at 7° C.

### Example 5

### Effect of Treatment of Soybeans with Marasmius scorodonius Peroxidase During a Bean Soaking Step and Effect of Further Contact with Marasmius Scorodonius Peroxidase after the Bean Soaking Step

### Pretreatment

Treated soybeans; 125 grams dehulled (i.e. hulls were removed) soybeans were mixed with 250 ml. water+MSP 2.25 DBLU/ml and 0.2 mM H₂O₂
Untreated soybeans; 125 grams dehulled soybeans were mixed with 250 ml. water Soybeans were stored in overnight at 7° C. in a dark environment.
Bleaching experiments were performed in fresh intermediate soy milk product prepared with a Vegan Star soymilk home cooker.

Sample Codes:

1. Soymilk of pretreated soybeans+MaxiBright 2.25 DBLU/ml.+0.2 mM H₂O₂

2. Soymilk of pretreated soybeans+0.2 mM H₂O₂

3. Reference soymilk of pretreated soybeans

4. Soymilk of untreated soybeans+MaxiBright 2.25 DBLU/ml.+0.2 mM H₂O₂

5. Soymilk of untreated soybeans+0.2 mM H₂O₂

6. Reference soymilk of untreated soybeans

### Results

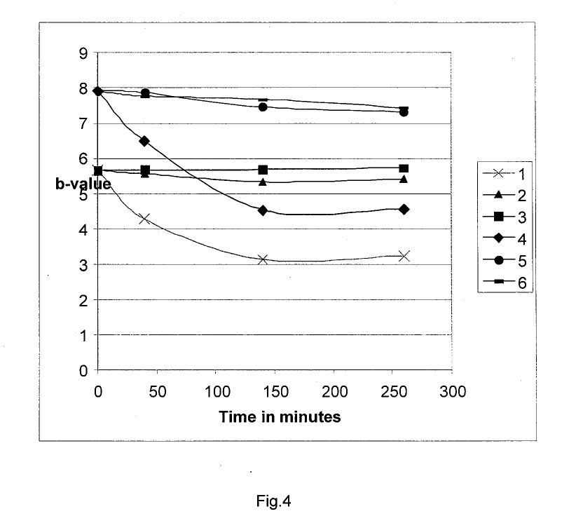

The results of soy milk bleaching are presented in FIG. 4 where b component of color was measured in time.

As can be seen from FIG. 4 the b-value of the color of soy milk prepared with pretreated soybeans is lower than the b-value of the color of soy milk of untreated soy beans, also resulting in a lower b-value after bleaching in the second step.

### REFERENCES

Schlich, P. (1993). Risk tables for discrimination tests. Food Quality and Preference, Volume 4, Pages 141-151.
Per Brockhof (2003). The statistical power of replications in difference tests, Food Quality and Preference, Volume 14, Pages 405-417.

1. A method for improving at least one characteristic of a bean-based product comprising contacting a bean-based product and/or an intermediate thereof with a Marasmium scorodonius peroxidase and/or a functional equivalent thereof and optionally preparing from said intermediate a bean-based product.

2. A method according to claim 1, wherein said bean-based product and/or an intermediate thereof is contacted with said peroxidase for a period of 1 minute to 5 days.

3. A method according to claim 1, wherein said bean-based product and/or an intermediate thereof is contacted with said peroxidase at a temperature from 2 to 90 degrees Celsius.

4. A method according to claim 1, wherein said bean-based product and/or an intermediate thereof is contacted with said peroxidase and hydrogen peroxide.

5. A method according to claim 4, wherein said hydrogen peroxide is added in an amount from 0.1-300 mg per liter bean-based product and/or an intermediate thereof.

6. A method according to of claim 1, wherein said bean-based product and/or an intermediate thereof is contacted with said peroxidase and an oxidoreductase.

7. A method according to claim 1, wherein said bean-based product and/or an intermediate thereof is contacted with said peroxidase, an oxidoreductase and a substrate for said oxidoreductase.

8. A method according to claim 1, wherein said bean-based product and/or an intermediate thereof is a soy-based product and/or an intermediate thereof.

9. A method according to claim 8, wherein said soy-based product and/or an intermediate thereof is soy milk, soy cheese, soy drink, soy blend and/or a fruitdrink comprising soy milk and/or a soy-based dairy alternative.

10. A method according to claim 1, wherein said characteristic is taste, smell, colour (whiteness), viscosity, and/or (soy) bean taste.

11. A method according to claim 6, further comprising inactivating said peroxidase and/or said oxidoreductase.

12. A method according to claim 1, wherein said peroxidase is contacted with a bean-based product and/or an intermediate thereof after a bean soaking step.

13. A method according to claim 1, wherein said peroxidase is contacted with a bean-based product and/or an intermediate thereof during a bean soaking step.

14. A method according to claim 1, wherein said peroxidase is contacted with a bean-based product and/or an intermediate thereof during and after a bean soaking step.

15. A method according to claim 1, wherein at least two characteristics of a bean-based product are improved.

16. A method according to claim 15, wherein said at least two characteristics are colour and taste.

17. A bean-based product obtainable by a method according to claim 1.

18. A Marasmius scorodonius peroxidase or a functional equivalent thereof that is capable of improving at least one characteristic of a bean-based product and/or an intermediate thereof.

19. A dual chamber package comprising in a first chamber a combination of a Marasmius scorodonius peroxidase and/or a functional equivalent thereof and an oxidoreductase and in a second chamber a substrate for said oxidoreductase, and/or in a first chamber a combination of Marasmius scorodonius peroxidase and/or a functional equivalent thereof and a substrate for an oxidoreductase and in a second chamber an oxidoreductase.

20. A vacuum packed composition of a Marasmius scorodonius peroxidase and/or a functional equivalent thereof, an oxidoreductase and a substrate for said oxidoreductase.

21. A composition comprising a Marasmius scorodonius peroxidase and/or a functional equivalent thereof, an oxidoreductase and a substrate for said oxidoreductase which composition has a reduced water activity.

22. A kit comprising a Marasmius scorodonius peroxidase and/or a functional equivalent thereof, an oxidoreductase and optionally a substrate for said oxidoreductase.
